(19) United States
(12) Patent Application Publication
(10) Pub. No.: US 2002/0082459 A1
(43) Pub. Date: Jun. 27, 2002

(54) High purity beta-carotene and process for obtaining same

(76) Inventors: David T. Bailey, Boulder, CO (US); Randall J. Daughenbaugh, Longmont, CO (US); Robert Arslanian, Pacifica, CA (US); Leonard A. Kaufmann, Denver, CO (US); Steven L. Richheimer, Broomfield, CO (US); Zhengjie Z. Liu, Superior, CO (US); James M. Piffarerio, Louisville, CO (US); Chris J. Kurtz, Boulder, CO (US)

Correspondence Address: Steven C. Petersen, Hogan & Hartson, LLP, Suite 1500, 1200 17th Street, Denver, CO, 80202, US

(21) Appl. No.: 10/001,880

(22) Filed: Nov. 16, 2001

Related U.S. Application Data
(63) Continuation of application No. 08/864,103, filed on May 28, 1997.

(51) Int. Cl. C07C 403/00

(52) U.S. Cl. 585/351

(57) ABSTRACT

The process of the present invention relates to the isolation and purification of both a natural mixed carotenoid product and an all-trans-β-carotene product from various different biomass sources, preferably from algae of the genus Dunaliella. More particularly, the present invention relates to a single solvent process whereby both natural and colorant products lie along the same process line. The nutritional product contains the natural array of carotenes and xanthophylls found in the plant material, while the colorant product contains primarily trans-β-carotene.

### CROSS-REFERENCE TO RELATED APPLICATIONS

This application is a Continuation of U.S. patent application Ser. No. 08/864,103, filed May 28, 1997, and entitled “High Purity P-Carotene and Process for Obtaining Same.”

### BACKGROUND OF THE INVENTION

1. Field of the Invention

The present invention relates to a process for the isolation and purification of both a natural mixed carotenoid product and an all-trans-β-carotene (TBC) product from a number of different biomass sources. More particularly, the present invention relates to a single solvent process whereby both nutritional and colorant products lie along the same process line.

2. Description of the State of Art

Carotenoids are the most widespread class of naturally occurring pigments in nature, present without exception in photosynthetic tissue and occurring with no definite pattern in non-photosynthetic tissues such as root, flower petals, seeds and fruits. They are also found in algae, fungi, yeasts, molds, mushrooms and bacteria, and in many cases they are the major pigment in the exoskeleton of aquatic and avian species. Carotenoids and/or carotenes derive their names from the fact that they constitute the major pigment in the carrot root, one of the first foods observed to possess this class of pigments.

Carotenoids are probably best generally described as aliphatic, aliphatic-alicyclic, or aromatic structures composed of five-carbon isoprene groups, usually eight, so linked that the two methyl groups nearest the center of the molecule are in positions 1 and 6 and all other lateral methyl groups are in positions 1 and 5. A series of conjugated C-C double bonds constitutes the chromophoric system. The carotenoids are subdivided into acyclic, monocyclic, and bicyclic derivatives, and respective parent compounds of each of the above categories are lycopene, γ-carotene, and β-carotene. The prefix “neo” is used to designate carotenoid stereoisomers containing at least one cis configuration in the double-bond chain, the prefix “pro” to designate some poly-cis-carotenoids, and the prefix “apo” to designate a carotenoid that has been derived from another carotenoid by loss of a structural element through degradative action.

All-trans-β-carotene, shown in FIG. 1, is generally considered as a class prototype. Beta-carotene is a symmetrical molecule of 40 carbon atoms, consisting of 8 isoprene units, having 11 conjugated double bonds, and possessing two β-ionone rings at the ends of the molecule. As will be discussed in further detail below the carotenoids, and specifically β-carotene are of particular importance not only because they represent a major dietary source of vitamin A, but also because they serve as excellent colorants and are the most prevalent in nature

Nutritional Role of Carotenes

The main function of carotenoid pigments in man is a nutritional one: that of providing a source of vitamin A. Vitamin A or retinol has long been known to be necessary to the biochemistry of vision and to the proper function of the epithelial tissues. Deficiencies of vitamin A may lead to reduced visual sensitivity, such as, night blindness and in extreme cases complete blindness or reduced resistance to infection through epithelial surfaces.

While vitamin A may be administered directly to an individual, there is a limited bodily tolerance to vitamin A, and overdoses can lead to toxic effects. It is thus significant that the enzymatic processes in the liver, which convert carotenes to vitamin A, produces only the amount of vitamin A that can be utilized by the body; an overdose is not produced. Consequently, an individual can be administered doses of carotene in quantities large enough to produce optimum levels of vitamin A in the body without risk of a toxic vitamin A reaction. Excess administered β-carotene is stored in fatty tissues and organs. Since the concentrations of β-carotene in the edible plants is relatively low, large quantities of plants must be consumed, or else the β-carotene must be supplied as a dietary supplement.

It is now known that β-carotene's function as a surrogate for vitamin A is not its only role. According to reports and clinical studies, β-carotene may be an important chemopreventive or chemo-postponing agent of promise in aging, immune deficiency, senile cataracts, and in several other types of cancer.

Carotenoids as Food Colorants

Color is one of the most significant properties of food to most consumers. The color of food is a significant factor in determining its acceptability. Consumers decisions about whether or not to purchase food are largely based on color. Color serves as an early signal of the inherent qualities of a food, such as freshness, spoilage, readiness for consumption, or as a sign of immaturity, thus creating a priori color-taste expectancy relationship. Consequently, there has always been and always will be a desire for attractively colored foods as long as the eye signals the selection of the daily ingestion of food products for the stomach via the brain. It would seem to follow, therefore, that the food industry will continue to require a vast array of acceptable, safe food colorants to satisfy consumer preferences. It is estimated that worldwide, the potential market for food colors may eventually reach several hundred million dollars or more annually.

The use of coloring agents to make food more attractive dates back to the early 1800's with the development of the food processing industry. Hundreds of coal-tar dyes were synthesized by 1900, of these, seven were selected as being physiologically harmless and suitable for food use. Due to safety reasons, however, only two of the seven coal-tar dyes are permitted to be in wide usage.

There appears to be a growing preference for natural-type colors in countries and by consumers around the world. The new color list of Switzerland distinguishes between colors occurring naturally in food and colors not naturally occurring in foods, and, in Norway, artificial colors may no longer be used. In Sweden, the use of artificial colors has been reduced to special cases only. Iceland has also established tighter controls over color additives to foods.

In general the all-trans-β-carotene is much more valuable than any of the cis-isomers, and is largely the only isomer of any commercial value. To date, all β-carotene used as a food colorant is synthetic; however, as consumers become increasingly more nutrition- and health-minded, a growing interest is developing in what is present in the food supply and particularly what is added to it in the way of food additives. Food labeling has increased this interest and there is a trend afoot, in which consumers want to avoid unfamiliar compounds that comprise food additives, such as antioxidants, preservatives and colors. In an attempt to avoid the consumption of synthetic compounds consumers easily adopt the concept that if an additive is in natural food it must be safe and good.

To meet the growing commercial markets in the “natural” nutritional and coloring industries, a number of methods have been proposed to isolate and purify β-carotenes. Few procedures if any, however, have successfully overcome the considerable obstacles posed by the need to prepare compounds of high purity from natural sources in an economical mauner while maintaining acceptability to the consumer and regulatory agencies.

A variety of different procedures for isolating and purifying β-carotenes from plant materials have been published. In the case of extracting β-carotene from palm oil, the known methods can be classified as follows:

(a) Extraction by saponification, wherein the palm oil is saponified to give soap, glycerol and a nonsaponifiable fraction containing carotenes. For examples of such, see Patent Application Nos: GB 657,682; U.S. Pat. No. 2,460,796; U.S. Pat. No. 2,440,029; US 2,572,467; and U.S. Pat. No. 2,652,433.

(b) Iodine method, wherein iodine is added to a solution of palm oil in petroleum ether, an insoluble precipitate of carotene di-iodides is formed. The iode compounds when treated with sodium thiosulfate however yield iso-carotenes or dehydrocarotenes which are not natural.

(c) Urea process, wherein triglycerides are broken down to fatty acids and methyl esters which then form insoluble compounds with urea thiourea, leaving the carotenoids in the remaining liquid.

(d) Extraction using Fuller's earth or activated carbon, wherein recovery of the carotenoids from the earth gives oxidized or isomerized carotenoids. For examples of such, see Patent Application Nos.: GB 691,924; GB 1,563,794; and U.S. Pat. No. 2,484,040.

(e) Extraction by selective solvents has been carried out using propane or furfural, see U.S. Pat. No. 2,432,021.

(f) Molecular distillation at 10-3 to 10-4 mm Hg. A process of trans esterification followed by molecular distillation of the ester. Fractions collected at 230° C. have a carotene content of about five times that of the original oil.

Liaaen-Jensen, S., The Carotenoids (O. Isler, ed), Birkhauser Verlag, Basel, p. 61 (1971), and Britton, G., Methods Enzymol., 111:113 (1985) described the extraction of carotenoids from plant and animal tissues. In brief, oxygen, light and heat are the most destructive factors and should be carefully avoided. The presence of oxygen during extraction may result in the formation of oxidative artifacts, or the disappearance of compounds, such as, phytofluene, due to complete oxidative breakdown. Furthermore, light and heat may cause isomerization. Peroxide-free solvents and an antioxidant such as butylated hydroxytoluene (BHT) should always be used during the extraction of carotenoids. If possible, exposure to acid and alkali (except for saponification) should also be avoided.

U.S. Pat. No. 4,680,314 to Nomura et al., discloses a process for concentrating algae and extracting β-carotene with an edible oil such as vegetable oil at elevated temperatures, that is, 66° to 100° C. The carotene concentration in the oil extract was reported to be on the order of 1.9%.

U.S. Pat. No. 4,439,629 to Ruegg et al., discloses a process for treating algae with calcium hydroxide at an elevated temperature to saponify the chlorophyll and produce a residue which is then filtered, dried, and extracted with a solvent, such as a halogenated hydrocarbon or an aliphatic or aromatic hydrocarbon, and recrystallized to yield enriched all-trans-β-carotene.

The above technical papers and patents are just a few examples of the many processes that currently exist in the literature, whereby β-carotenes are extracted and isolated from various plant materials. However each process disclosed involves multiple steps using various solvents. Consequently, the disclosed processes are not easily scaled up to an efficient commercial process where disposal considerations of various solvents play an important role in the overall feasibility of the process.

A further disadvantage of the processes disclosed in the literature is the inability to achieve a high concentration and purity level of the all-trans-β-carotene isomer. A number of methods have been developed to convert the cis-carotenoids to all-trans-carotenoids; however, these methods utilize synthetic starting materials and/or are unable to yield a pure all-trans-β-carotene product. Invariably a small amount of cis-isomers are present as contaminants in the final product. See, U.S. Pat. Nos. 2,849,507; 3,441,6233; and 3,989,757.

There is still a need, therefore, for a process and procedure for isolating and purifying natural carotenoids for nutritional use and further enhancing for and purifying the all-trans-β-carotene for use as a natural colorant.

### SUMMARY OF THE INVENTION

Accordingly, it is an object of the present invention to provide a simplified method for the extraction, isolation and purification of carotenoid compounds.

It is a further object of the present invention to provide a single solvent process whereby both nutritional and colorant products lie along the same process line.

It is also an object of the present invention to increase the yield of all-trans-β-carotene.

Additional objects, advantages and novel features of this invention shall be set forth in part in the description that follows, and in part will become apparent to those skilled in the art upon examination of the following specification or may be learned by the practice of the invention. The objects and advantages of the invention may be realized and attained by means of the instrumentalities, combinations, and methods particularly pointed out in the appended claims.

To achieve the foregoing and other objects and in accordance with the purposes of the present invention, as embodied and broadly described therein, the method of this invention comprises contacting a plant material containing carotenoids with a solvent thereby forming an extract that is subsequently filtered and heated so as to evaporate off substantially all of the solvent resulting in a mixture of carotenoids. The mixture of carotenoids can be further isomerized to obtain an all-trans-β-carotene.

The present invention is also directed to a composition of naturally obtained all-trans-β-carotene having a purity level greater than 98%.

### BRIEF DESCRIPTION OF THE DRAWINGS

The accompanying drawings, which are incorporated in and form a part of the specification, illustrate the preferred embodiments of the present invention, and together with the descriptions serve to explain the principles of the invention.

In the Drawings:

FIG. 1 is a structural representation of β-carotene.

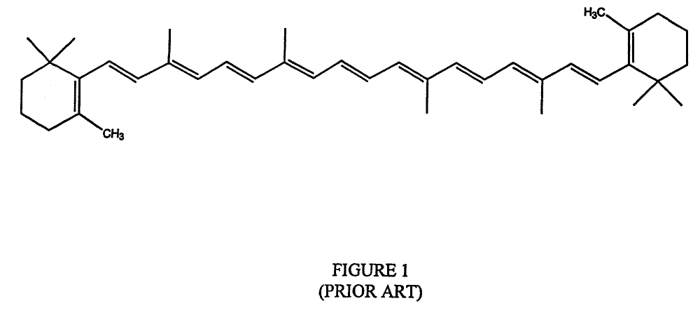

FIG. 2 is a graphical representation of the percentage change in trans-β-carotene due to isomerization of cis-β-carotene compounds at three different temperatures.

FIG. 3 is a graphical representation of the time required to reach the maximum ratio of trans to cis isomers when the temperatures are stacked in accordance with the present invention.

FIG. 4 is the experimental data of Example 1 representing the percentage change in trans-β-carotene due to the isomerization of cis-β-carotene.

### DETAILED DESCRIPTION OF THE INVENTION

In general the present invention relates to a single solvent process whereby both a natural mixed carotenoid product and an all-trans-β-carotene colorant product lie along the same process line. The nutritional product contains the natural array of carotenes and xanthophylls found in the plant material, while the colorant product contains primarily trans-β-carotene. The process includes contacting a plant material that contains β-carotene with a solvent, thus resulting in a crude extract containing a mixture of compounds that includes carotenoid compounds. The crude extract is filtered to remove suspended fine plant materials and then heated to evaporate substantially all of the solvent resulting in an oil. The oil may be used as a nutritional product or as a precursor to a colorant product. If a colorant product is desired, the oil is further heated, thereby isomerizing the cis-β-carotene compounds to all-trans-β-carotene isomers. The all-trans-β-carotene compounds are then crystallized with the addition of cold solvent.

The β-carotene containing compositions of the present invention may be prepared from a variety of plant materials, such as algae, palms, vegetables such as spinach, broccoli, alfalfa, and other plants. Preferably the plants are algae. Among the algae, the preferred classes are Chlorophyta (green algae), of which the preferred genus is Dunaliella. Other genera may also be used so long as carotene can be produced in relatively large quantities. Cultivation techniques may significantly increase the amount of carotene present in each algal cell or body.

Typically, the algae are raised in shallow tanks, bioreactors, man-made or natural ponds at a wide range of temperatures, such as from 15 to 50° C., and more preferably from about 25 to 45° C. Preferably the culture medium is salt water, but fresh water can also be used. Fresh water may be made saline by the addition of salt as a culture medium. The medium may be supplemented by the addition of nitrate, phosphate, bicarbonate, iron and trace minerals. Protocols for the large scale propagation of algae are described in, for example, Richmond, A., Handbook of Microalgal Mass Culture, CBC Press, Boca Raton, Fla., (1986), and Ben-Amotz, A., Algal and Cyanobacterial Biotechnology, Longman Scientific and Technical Press, pp. 90-114, (1989), both of which are incorporated herein by reference. When the algal culture reaches the desired density, such as about 0.25 to 0.5 grams dry weight/liter, as determined by absorbance, the algae are harvested from the tank or pond by pumping out the water slurry containing the dispersed algae. The slurry may be passed through screens which are sufficiently coarse to allow algae through but which remove larger unwanted objects.

In the preferred embodiment, the slurry is dewatered and concentrated by centrifugation, evaporation, flocculation, dispersed air flotation, etc. Following this concentration step, the emulsifying agents, such as glycerol, are removed from the algal material using ultra-filtration. The algal material is pumped through a fill valve into a feed tank which is connected in a closed loop system to an ultra-filter. When the feed tank has the required amount of algal material, the fill valve is closed and the algal material is pumped through the ultra-filter at temperatures in the range of 60 to 70 ° C. When the algal material has been concentrated to half its original volume, filtered fresh water is added to bring the algal material back to its original volume. The fresh water is filtered through 10 μm and 0.2 μm filters prior to being added to the algal concentrate. The process is repeated again, and preferably three more times for a total of four washes. The fresh water washes remove the salt and water solubles from the algal material. The ceramic membrane pore sizes used in the ultra-filtration unit are in the range of 0.09-0.5 μm, and preferably 0.1 μm. The performance of the 0.1 μm filter is comparable to the 0.5 μm filter, but it is less likely to plug with algal solids. The cleaning and sterilization procedure entitled “MAMBRALOX® Ceramic Membrane Modules” described by US Filter, United States Filter Corporation was followed, and is hereby incorporated by reference. The carotenes are then extracted from the ultra-filtered algal material or other plant preparation by use of a suitable organic solvent. The extraction and subsequent purification procedures are typically performed under low light intensity and under vacuum or an atmosphere of inert gas (e.g., nitrogen) to maximize recovery of non-oxidized carotenes. The extraction solvent used in the present invention is heptane, a non-acidic solvent.

In the extraction step, the temperature of extraction is between 25 to 100° C., with 45 to 60° C. being preferred. The amount of algal material to solvent mixture used in the extraction process varies between 1:30 to 1:3,000 on a gram to milliliter basis, with 1:200 to 1:400 being preferred. The plant material prior to the addition of solvent typically contains in the range of 100 to 900,000 ppm of solvent and preferably 0 to 70,000 ppm. Carotenoid extraction is carried out in a container, preferably a baffled container, using an overhead, high shear mixer, such as a Lightnin Lab Mixer (Model No. LIU08F manufactured by Lightnin) at 0.070 to 0.4 hp/gal for a time period of 10 minutes to five hours, with 20 minutes to 60 minutes being preferred. The resulting extract is allowed to stand for a period of time sufficient to form two phases. The top organic phase, containing the carotenoids, is removed and saved whereupon an equivalent volume of fresh solvent is added to the lower phase and the extraction sequence is repeated. Again the extract is allowed to stand for a period of time sufficient to achieve the formation of two phases. The top organic phase is removed and pooled with the prior organic phase.

The pooled organic phase is then filtered in vacuo through a filter having a pore size in the range of 0.5-100 μm, and preferably 10 μm. Whatmann #1 filter paper is preferred. The temperature of the organic phase prior to filtration is between −20 to 100° C., with room temperature being preferred. The filtrate, which contains the mixed carotenoids is heated to a temperature between 80 and 100° C., and preferably 98° C. to remove most of the solvent resulting in the oil intermediate. Alternatively, the filtrate is concentrated under reduced pressure at a temperature of about 50° C. to produce a substantially solvent free oil. The still hot oil intermediate is transferred to a vacuum oven, preheated in the range of 80 and 100° C. and preferably 98° C., for a period of time sufficient to remove the residual solvent. Typically the residual solvent will be removed in 1 to 72 hours, with 16 hours being preferred. The resulting reddish oil product contains 30% to 40% carotenoids by weight and has less than 100 ppm residual solvent as measured using GC/MS head space analysis. This oil comprising both cis and trans isomers of β-carotene is suitable as a nutritional product, or the resulting oil can be used as an intermediate in the production of a high purity all-trans-β-carotene product which may be used as a natural food colorant.

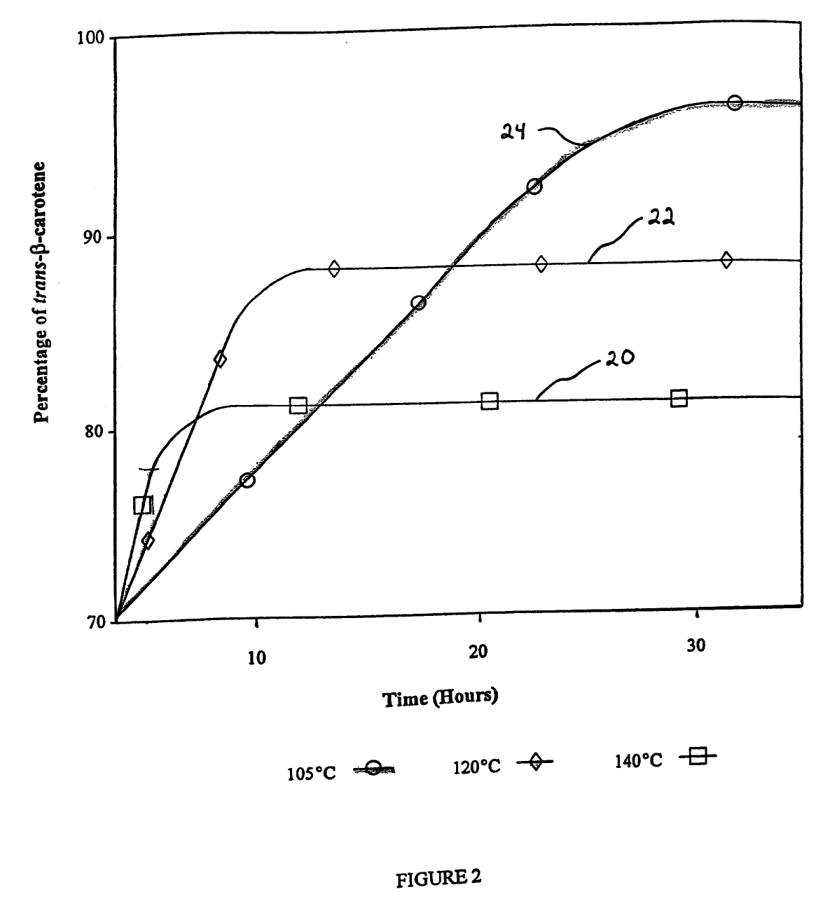

As is demonstrated in FIG. 2, the equilibrium ratio of the trans-β-carotene and cis-β-carotene isomers is temperature dependent. In theory, the cis isomers contained in the oil from the previous step if kept at room temperature would ultimately be converted to the trans form; however, this conversion or isomerization would take months or possibly years. However, when a carotenoid mixture having 70% trans-β-carotene and 30% cis-β-carotene is heated to 140° C., approximately 11% of the cis-β-carotene isomers will ultimately be converted to the trans isomer form. Consequently, this trans:cis isomer equilibrium, represented by curved line 20, is reached at approximately 81% trans-β-carotene to 19% cis-β-carotene. However, when the same mixture is heated to 120° C. the trans:cis equilibrium, represented by curved line 22, is reached at approximately 88% trans-β-carotene to 12% cis-β-carotene. Finally, when the heat is reduced to 105° C., 26% of the cis-β-carotene isomers are converted to the trans form, represented by curved line 24. Consequently, depending upon the desired percentage of trans-β-carotene, the temperature can either be raised, thereby yielding a low percentage of trans-β-carotene in a short period of time or lowered, thereby yielding a high percentage of trans-β-carotene but over a long period of time.

The final step in the process of the present invention, the isomerization step, subjects the reddish oil from the previous step to a temperature in the range of about 90° C. to 140° C. and preferably in the range of 100° C. to 120° C. in an inert atmosphere for a period of time sufficient to result in the isomerization of the cis-β-isomers. Preferably, heating of the oil is carried out for approximately 15 to 35 hours.

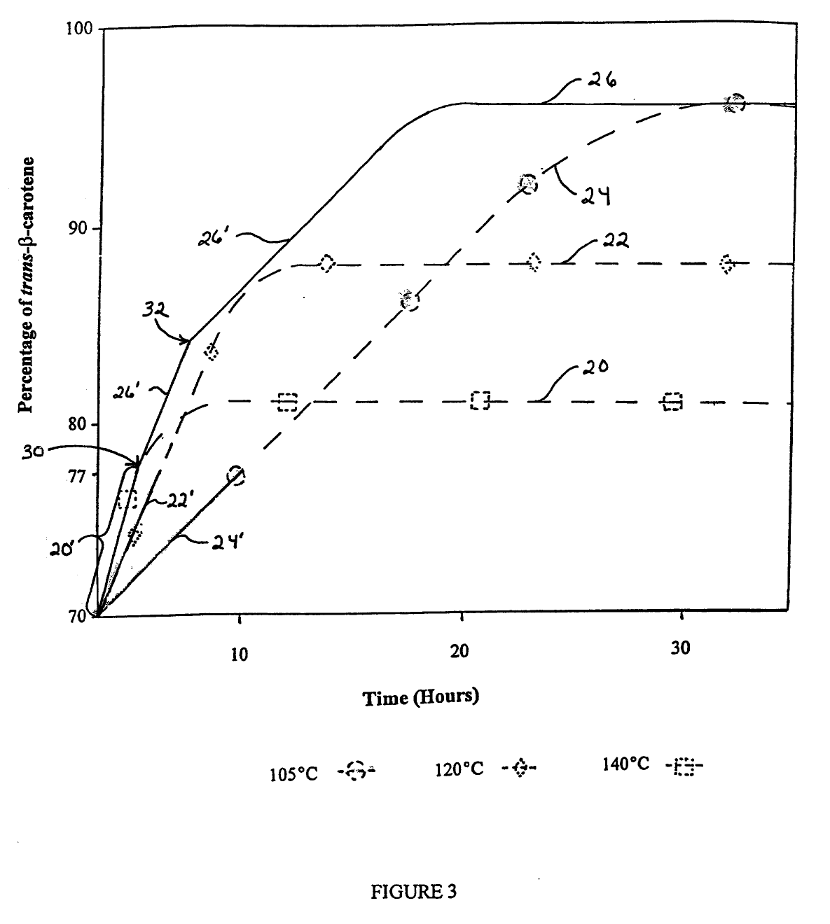

In an alternate embodiment, shown in FIG. 3, the time required to reach the maximum equilibrium, represented by curved line 26, is substantially decreased by stacking the linear isomerization rates of discrete temperatures. It should be noted that while temperature defines the equilibrium ratio of trans-β-carotene to cis-β-carotene, the rate at which this ratio increases occurs much more rapidly at higher temperatures than it does at lower temperatures, that is, approximately 7% of the cis isomers will be converted to the trans form in approximately 2½ hours at 140° C., shown as the straight line 20′ versus 3¾ hours at 120° C., straight line 22′ and approximately 10 hours at 105° C., straight line 24′. FIG. 3 is illustrative of the results obtained by stacking only three temperatures, that is, 140° C., 120° C., and 105° C. At knee 30, FIG. 3, the 140° C. time period ceases and the 120° C. time period begins, and knee 32 represents the end of the 120° C. time period and the beginning of the 105° C. time period. Curved line 26 would obviously be optimized if all the possible time periods between 140° C. and 105° C. were plotted.

Consequently, the second embodiment of the isomerization step of the present invention contemplates subjecting the oil from the previous step to a starting temperature of approximately 140° C., and then gradually reducing the temperature at a rate that maintains an optimum rate of isomerization until the desired equilibrium of trans :cis isomers is reached. This may be accomplished by placing the oil in an insulated tank at a starting temperature of approximately 140° C. However, the starting temperature will be dependent on the percentage of trans-β-carotene in the starting material, that is, if the percentage of trans-β-carotene is greater than approximately 77% the starting temperature will be reduced accordingly. The tank is then purged of air by filling it with an inert gas, such as argon, and the temperature of the tank is then gradually reduced so as to maintain an optimum rate of isomerization, represented by line 26′.

The isomerized product is then washed twice with a solvent such as heptane at a temperature of −15° to 25° C. to remove all soluble impurities resulting in a product suitable for use as a colorant product. The wash at a lower temperature causes the all-trans-β-carotene isomers to crystallize and fall out of solution. Surprisingly, the crystallization in combination with the isomerization allows for an overall recovery of approximately 130% (with respect to the initial amount of trans-β-carotene). Even more surprisingly, from the crude oil extract a purity level of greater than 98% is achieved.

The following non-limited examples provide specific high yield, high purity processes for isolating and purifying carotenes from plant tissues. All scientific and technical terms have the meanings as understood by one with ordinary skill in the act. Carotenoid recovery was assayed using the YMC3 HPLC method. HPLC was measured on a Hitachi 2000 spectrophotometer. Commercially available chemicals were used without any further purification.

### EXAMPLE 1

### Preparation of trans-β-carotene for use as a Colorant Product

A. Extraction of Carotenoid

To 400 g ultra-filtered algal material 1600 ml of heptane, preheated to 50° C., was added. The components in a 4 L baffled beaker were mixed at 1800 rpm for 20 minutes using a Lightnin Lab Mixer with a combination of high shear and high flow impellers. A water bath heated to 50° C. was used to maintain temperature throughout the extraction. As demonstrated in Tables 1, 2 and 3 below, the types of impellers, the mixing time, and the mixing speed all have an important impact on the β-carotene (BC) recovery.

TABLE 1
Extraction-Impeller Comparison

| | | Extrac- | | Extrac- |
|---|---|---|---|---|
| | Extraction 1: | tion 2: | Extraction 3: | tion 4: |
| | BC Recovery | BC Re- | BC Recovery | BC Re- |
| Impeller Type | % | covery % | % | covery % |
| Marine | 49 | 77 | 89 | 95 |
| Sawtooth | 70 | 90 | 94 | 96 |
| Combination | 68 | 90 | 94 | 96 |

TABLE 2
Extraction-Mixing Time Comparison

| | Mixing | | Mixing | |
|---|---|---|---|---|
| | Time: 4 | Mixing Time: | Time: 15 | Mixing Time |
| | minutes BC | 10 minutes | minutes BC | 20 minutes |
| Power | Recovery | BC Recovery | Recovery | BC Recovery |
| 1711-127 | 77% | 82% | 90% | 93% |

TABLE 3
Extraction-Mixing Speed Comparison

| | Temperature | % Heptane | |
|---|---|---|---|
| Experiment | after mixing | Recovery | % BC Recovery |
| 3.0 K rpm | 30° C. | 97 | 75 |
| 4.7 K rpm | 30° C. | 96 | 86 |
| 6.0 K rpm | 34° C. | 96 | 93 |

The baffled beaker was allowed to stand for 30 minutes before the top heptane extract was decanted. In this manner a total of four extractions were carried out. The extracts and spent algal material were assayed and organic layers pooled. The β-carotene extract pool was filtered through a Whatmann #1 filter paper and assayed using the YMC3 HPLC method disclosed in a YMC Technical Data Bulletin, titled “Carotenoid Column,” YMC, Inc., Wilmington, N.C., and incorporated herein by reference.

B. Evaporation

Four liters of β-carotene extract were concentrated by rotary evaporation to remove the heptane, in continuous feed mode, at 50° C. to an oil. A small amount of heptane (about 20 mL) was added back to the 2 L rotary evaporation flask in order to facilitate transfer to a tared 100 mL round bottom. The mixture was again concentrated to an oil at 50° C. by rotary evaporation to remove the heptane. The contents of the flask were assayed and the results are represented in Tables 4 and 5 below.

TABLE 4
Evaporation-Residual Heptane

| | Residual | Carotenoid | Carotenoid | Mass |
|---|---|---|---|---|
| | Heptane | Purity | Recovery | Balance |
| Material | (ppm) | (% in oil) | (%) | (%) |
| Starting Material | 60000 | 35 | | |
| (algae oil) | | | | |
| Low heptane | <100 | 42 | 100 | 100.3 |
| mixed carotenoids | | | | |

TABLE 5
Evaporation-Carotenoid Profile

| | | | cis-beta- | trans-beta- | trans-alpha |
|---|---|---|---|---|---|
| Material | Lutein | zeaxanthin | carotene | carotene | carotene |
| Starting | 1.2 | 0.4 | 49.5 | 45.3 | 3.6 |
| material | | | | | |
| Low heptane | 1.3 | 0.3 | 50.7 | 43.2 | 4.4 |
| Mixed | | | | | |
| Carotenoids | | | | | |

This produced an algae oil containing less than 100 ppm residual heptane, suitable for use as a nutritional product or as an intermediate in colorant production.

C. Cis/trans Isomerization

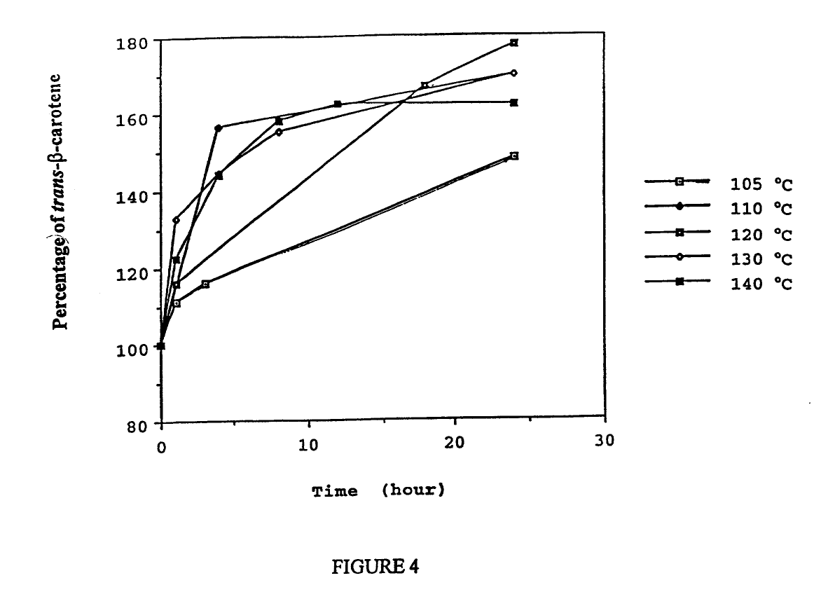

The algae oil (2.73 g) was weighed into a dried (100° C. for 5 hours) tared 10 ml round bottom flask. The flask was purged of air by filling with argon for about 0.5 hours. The oil was heated to 120° C. for 24 hours with stirring under an inert atmosphere. The purpose of the isomerization step was to increase the yield of trans-β-carotene (TBC) in route to the colorant product. In a separate experiment, the results of which are shown in FIG. 4 and Table 6 below, isomerization of 13 and 9-cis-β-carotene to trans-β-carotene was found to occur at temperatures between 105° to 140° C. although significant degradation was found to occur at 140° C.

TABLE 6
Isomerization-TBC Recovery

| | TBC Recovery % | Carotenoid Loss % |
|---|---|---|
| Experiment | @ 24 hour | @ 24 hour |
| 105° C. | 148 | 0 |
| 110° C. | 170 | 0 |
| 120° C. | 190 | 4 |
| 130° C. | 142 | 11 |
| 140° C. | 88 | 33 |

It was determined that 120° C. gave the highest trans-β-carotene recovery at 190% after 24 hours with only 4% loss of total carotenoids.

D. Heptane Wash Step

To 2.5 g of the isomerization product was added 7 ml of cold heptane C (−10° C.). The material was stirred with a spatula and then chilled to −20° C. for 1 hour. The material was filtered and washed three times with 13 ml of cold heptane (−10 to −5° C.). The purpose of the washes is to remove all heptane soluble impurities. Table 7 below demonstrates that three washes are adequate to remove all soluble impurities.

TABLE 7
Colorant Wash-Impurity Removal Data

| | % Carotenoid | |
|---|---|---|
| Experiment | Impurity Removal | % Trans beta-carotene loss |
| First Wash | 95 | 6 |
| Second Wash | 98 | 1 |
| Third Wash | 100 | 1 |

The crystals were dried overnight (17 hours) in a vacuum oven (50° C.). Table 8 shows the data from eight different experiments for trans-β-carotene recoveries for use as colorant products. The results from this Example 1 are given in the first line of the table.

TABLE 8
Trans-beta-Carotene Recoveries for Colorant Product

| | Extraction | Isomer. | Wash | Total |
|---|---|---|---|---|
| | Recovery* | Recovery* | Recovery* | Recovery* |
| Experiment | (%) | (%) | (%) | (%) |
| Example I | 98 | 144 | 91 | 128 |
| | 97 | 150 | 97 | 141 |
| | 97 | 146 | 95 | 135 |
| | 96 | 136 | 94 | 123 |
| | 97 | 127 | 84 | 103 |
| | 97 | 175 | 93 | 158 |
| | 98 | 148 | 86 | 125 |
| | 81 | 157 | 81 | 103 |
| Average | 95 | 149 | 90 | 127 |

*Recoveries are with respect to initial amount of trans-β-carotene

### EXAMPLE 2

### Preparation of Carotenoid Nutritional Product

A. Extraction of Carotenoid

To 0.5 g of algae oil containing 60,000 ppm heptane, 150 ml of technical grade heptace C preheated to 50° C. was added. The algae oil was dissolved with stirring using a magnetic stirrer. The solution, which was allowed to cool to room temperature, was filtered in vacuo throu Whatmann #1 filter paper. The filtrate, which contained the mixed carotenoids, was collected and stored in an amber bottle at room temperature.

B. Mixed Carotenoid Product

A 10 ml aliquot from the filtrate was transferred via pipette into a tared aluminum pan. The pan was placed inside a convection oven at 95° C. for 22 minutes. The pan was removed from the oven and placed directly into a vacuum oven at 95° C. for one hour. The oil was assayed for carotenoids and residual solvent by the GC/FIMD direct injection method.

C. Residual Heptane by Headspace Analysis

Four aliquot containing 10 ml of the filtrate were placed into tared vials. The heptane was removed by heating the containers to 95° C. for one hour. The remaining oil was quickly transferred into a vacuum oven at 95° C. where it was kept for 16 hours.

Results

The vials were weighed and spiked with 0-4 μL of heptane. Analysis by standard addition headspace GC/MS showed the algae oil to contain 65 ppm heptane C.

TABLE 9

| | Residual | Carotenoid | Carotenoid | Mass |
|---|---|---|---|---|
| | Heptane | Purity | Recovery | Balance |
| Material | (ppm) | (% in oil) | (%) | (%) |
| Starting Material | 60000 | 35 | | |
| (algae oil) | | | | |
| Low heptane mixed | Not Detected | 42 | 100 | 100.3 |
| carotenoids | | | | |

The carotenoid ratio before and after heptane evaporation was compared and found to be very similar.

TABLE 10

| | | | | trans- | trans- |
|---|---|---|---|---|---|
| | | | cis-beta- | beta- | alpha- |
| Material | Lutein | zeaxanthin | carotene | carotene | carotene |
| Starting material | 1.2 | 0.4 | 49.5 | 45.3 | 3.6 |
| Low heptane Mixed | 1.3 | 0.3 | 50.7 | 43.2 | 4.4 |
| Carotenoids | | | | | |

The foregoing description is considered as illustrative only of the principles of the invention. Furthermore, since numerous modifications and changes will readily occur to those skilled in the art, it is not desired to limit the invention to the exact construction and process shown as described above. Accordingly, all suitable modifications and equivalents may be resorted to falling within the scope of the invention as defined by the claims which follow.

The invention claimed is:

1. A single-solvent method of isolating and purifying all-trans-β-carotene from any plant material that contains carotenoids, wherein the same type of solvent is used in all steps utilizing a solvent, said method comprising:
(a) contacting said plant material for a selected period of time with said solvent, whereby said carotenoids are solubilized and transported into said solvent forming a crude extract;
(b) collecting and filtering said crude extract;
(c) evaporating said solvent from said crude extract thereby forming an oil containing said carotenoids, wherein said oil is substantially free of said solvent;
(d) heating said substantially solvent free oil for a sufficient period of time and at a sufficient temperature to isomerize said carotenoids capable of being isomerized to all-trans-β-carotene isomers; and
(e) washing said oil with said solvent, whereby the all-trans-β-carotene isomers are crystallized.

2. The method of claim 1, wherein said solvent is heptane.

3. The method of claim 1, wherein said plant material is an algae.

4. The method of claim 3, wherein said algae is Dunaliella salina.

5. The method of claim 1, wherein said selected period of time is from about 10 minutes to 5 hours.

6. The method of claim 4, wherein said selected period of time is from 20 to 60 minutes.

7. The method of claim 3, wherein said algae is treated prior to said contacting step to remove emulsifying agents.

8. The method of claim 7, wherein said emulsifying agents are removed by ultra-filtration.

9. The method of claim 1, wherein said filtering step utilizes a filter having a pore size in the range of 1 to 100 μm.

10. The method of claim 1, wherein said evaporation step occurs at a temperature in the range of 80 to 100° C.

11. The method of claim 10, wherein said temperature is about 98° C.

12. The method of claim 1, wherein said heating step occurs at a temperature of 105° to 140° C.

13. The method of claim 12, wherein said temperature is 120° C.

14. The method of claim 12, wherein said heating step requires 1 to 24 hours.

15. The method of claim 13, wherein said heating step requires about 24 hours.

16. The method of claim 1, wherein said heating step comprises:
(a) heating said substantially solvent free oil to a temperature of about 140° C. and maintaining said temperature at about 140° C. for about one hour;
(b) reducing said temperature to about 110° C. and maintaining said temperature at about 110° C. for about one hour; and
(c) reducing said temperature to about 105° C. and maintaining said temperature at about 105° C. for about six hours.

17. The method of claim 1, wherein said solvent in said washing step is at a temperature of about −15° to 25° C.

18. A single-solvent method of isolating and purifying all-trans-β-carotene from any algal material that contains carotenoids, wherein the same type of solvent is used in all steps utilizing a solvent, said method comprising:
(a) removing emulsifying agents from said algal material;
(b) extracting said carotenoid compounds from said algal material by mixing said algal material with a solvent, whereby said carotenoids are solubilized and transported into said solvent forming a crude extract;
(c) collecting and filtering said crude extract;
(d) evaporating said solvent from said crude extract by heating said crude extract to a temperature of about 80 to 100° C., thereby forming an oil substantially free of solvent;
(e) heating said substantially solvent free oil to a temperature of about 105° to 140° C. for about 1 to 24 hours to convert said carotenoids capable of being isomerized to all-trans-β-carotene isomers; and
(f) crystallizing said all-trans-β-carotene by washing said heated oil with said solvent, wherein said solvent for said washing is at a temperature of about −15° C. to 25° C.

19. The method of claim 18, wherein said emulsifying agents are removed by ultra-filtration.

20. The method of claim 18, wherein said algal material is Dunaliella salina.

21. The method of claim 18, wherein said solvent is heptane.

22. The method of claim 18, wherein said evaporation step occurs at about 98° C.

23. The method of claim 18, wherein said heating step occurs at about 120° C.

24. A process for converting a substantially solvent free cis-carotene isomer to an all-trans-carotene isomer, comprising:
(a) subjecting the substantially solvent free cis-carotene isomer to an initial temperature of approximately 140° C. and maintaining said temperature at about 140° C. for about one hour;
(b) reducing said temperature to about 110° C. and maintaining said temperature at about 110° C. for about one hour; and
(c) reducing said temperature to about 105° C. and maintaining said temperature at about 105° C. for about six hours.

25. The process of claim 24, wherein said reducing steps (b) and (c) take place at a rate that maintains an optimum rate of cis-carotene isomer to trans-carotene isomer conversion.

26. The process of claim 24, wherein said cis-carotene isomer is β-carotene.

27. A single-solvent process for making both a first mixed carotenoid oil product and a second all-trans-β-carotene product from any plant material that contains carotenoids, wherein the same type of solvent is used in all steps utilizing a solvent, said method comprising:
(a) contacting said plant material for a selected period of time with said solvent, whereby said carotenoids are solubilized and transported into said solvent forming a crude extract;
(b) collecting and filtering said crude extract;
(c) evaporating said solvent from said crude extract thereby forming an oil containing said first mixed carotenoid oil product, wherein said oil is substantially free of said solvent;
(d) heating said substantially solvent free first mixed carotenoid oil product for a sufficient period of time and at a sufficient temperature to isomerize the carotenoids in said first mixed carotenoid oil product capable of being isomerized to all-trans-β-carotene isomers; and
(e) crystallizing said all-trans-β-carotene isomers from said heated mixed carotenoid oil product by washing said heated mixed carotenoid oil product with said solvent, wherein said solvent is at a temperature of about −15° C. to 25° C., whereby said second all-trans-β-carotene product is isolated.

28. The process of claim 27, wherein said solvent is heptane.

29. The process of claim 27, wherein said plant material is an algae.

30. The process of claim 27, wherein said selected prior of time for contacting said solvent with said plant material is form about 10 minutes to 5 hours.

31. A single-solvent method of isolating and purifying carotenoids from any plant material that contains carotenoids, wherein the same type of solvent is used in all steps utilizing a solvent, said method comprising:
(a) contacting said plant material with said single, non-acidic extraction solvent for a selected period of time, whereby said carotenoids are solubilized and transported into said non-acidic extraction solvent forming a crude extract;
(b) collecting and filtering said crude extract; and
(c) evaporating said non-acidic, extraction solvent from said crude extract thereby forming an oil containing said carotenoids, wherein said oil is substantially free of said non-acidic extraction solvent.

32. The method of claim 31, further comprising:
(d) heating said oil from step (c) for a sufficient period of time and at a sufficient temperature to isomerize said carotenoids capable of being isomerized to all-trans-β-carotene isomers; and
(e) washing said oil from step (d) with said non-acidic extraction solvent, whereby the all-trans-β-carotene isomers are crystallized.

33. The process of claim 31, wherein said non-acidic extraction solvent is heptane.

34. The process of claim 31, wherein said plant material is an algae.

35. The process of claim 31, wherein said algae is Dunaliella salina.

36. The method of claim 31, wherein said selected period of time is from about 10 minutes to 5 hours.

37. The method of claim 36 wherein said selected period of time is from 20 to 60 minutes.

38. The method of claim 34, wherein said algae is treated prior to said contacting step to remove emulsifying agents.

39. The method of claim 38, wherein said emulsifying agents are removed by ultra-filtration.

40. The method of claim 31, wherein said filtering step utilized a filter having a pore size in the range of 1 to 100 μm.

41. The method of claim 31, wherein said evaporation step occurs at a temperature in the range of 80 to 100° C.

42. The method of claim 41, wherein said temperature is about 98° C.

43. The method of claim 32, wherein said heating step occurs at a temperature of 105° to 140° C.

44. The method of claim 43, wherein said temperature is 120° C.

45. The method of claim 43, wherein said heating step requires 1 to 24 hours.

46. The method of claim 44, wherein said heating step requires about 24 hours.

47. The method of claim 32, wherein said heating step comprises:
(a) heating said oil from step (c) to a temperature of about 140° C. and maintaining said temperature at about 140° C. for about one hour;
(b) reducing said temperature to about 110° C. and maintaining said temperature at about 110° C. for about one hour; and
(c) reducing said temperature to about 105° C. and maintaining said temperature at about 105° C. for about six hours.

48. The method of claim 32, wherein said extraction solvent in said washing step is at a temperature of about −15° to 25° C.

49. A single-solvent method of isolating and purifying all-trans-β-carotene from any plant material that contains carotenoids, comprising:
(a) contacting said plant material for a selected period of time with said solvent, whereby said carotenoids are solubilized and transported into said solvent forming a crude extract;
(b) collecting and filtering said crude extract;
(c) evaporating said solvent from said crude extract thereby forming an oil containing said carotenoids, wherein said oil is substantially free of said solvent;
(d) heating said substantially solvent free oil for a sufficient period of time and at a sufficient temperature to isomerize said carotenoids capable of being isomerized to all-trans-β-carotene isomers; and
(e) washing said oil with said solvent, whereby the all-trans-β-carotene isomers are crystallized, wherein the steps of this method all use the same solvent.

50. A method consisting of a single-solvent system for isolating and purifying carotenoids from a plant material containing carotenoids wherein the method comprises:
(a) contacting said plant material for a selected period of time with said solvent, whereby said carotenoids are solubilized and transported into said solvent forming a crude extract;
(b) collecting and filtering said crude extract;
(c) evaporating said solvent from said crude extract thereby forming an oil containing said carotenoids, wherein said oil is substantially free of said solvent;
(d) heating said substantially solvent free oil for a sufficient period of time and at a sufficient temperature to isomerize said carotenoids capable of being isomerized to all-trans-β-carotene isomers; and
(e) washing said oil with said solvent, whereby the all-trans-β-carotene isomers are crystallized.

51. A method for isolating and purifying carotenoids from a plant material containing carotenoids, comprising:
(a) utilizing only one single solvent throughout the entire method;
(b) contacting said plant material for a selected period of time with said solvent, whereby said carotenoids are solubilized and transported into said solvent forming a crude extract;
(c) collecting and filtering said crude extract;
(d) evaporating said solvent from said crude extract thereby forming an oil containing said carotenoids, wherein said oil is substantially free of said solvent;
(e) heating said substantially solvent free oil for a sufficient period of time and at a sufficient temperature to isomerize said carotenoids capable of being isomerized to all-trans-β-carotene isomers; and
(f) washing said oil with said solvent, whereby the all-trans-β-carotene isomers are crystallized.

52. A method for isolating and purifying carotenoids from a plant material containing carotenoids, comprising:
(a) contacting said plant material for a selected period of time with a solvent, wherein said solvent consists of the same type of solvent utilized throughout the entire method and whereby said carotenoids are solubilized and transported into said solvent forming a crude extract;
(b) collecting and filtering said crude extract;
(c) evaporating said solvent from said crude extract thereby forming an oil containing said carotenoids, wherein said oil is substantially free of said solvent;
(d) heating said substantially solvent free oil for a sufficient period of time and at a sufficient temperature to isomerize said carotenoids capable of being isomerized to all-trans-β-carotene isomers; and
(e) washing said oil with said solvent, whereby the all-trans-β-carotene isomers are crystallized.
